(19) United States
(12) Patent Application Publication
(10) Pub. No.: US 2014/0368827 A1
(43) Pub. Date: Dec. 18, 2014

(54) OPTICAL TOMOGRAPHY APPARATUS, OPTICAL TOMOGRAPHY METHOD, AND OPTICAL COHERENCE TOMOGRAPHY APPARATUS

(71) Applicant: CANON KABUSHIKI KAISHA, Tokyo (JP)

(72) Inventor: Eiichi Fujii, Kamakura-shi (JP)

(21) Appl. No.: 14/295,534

(22) Filed: Jun. 4, 2014

(30) Foreign Application Priority Data
Jun. 13, 2013 (JP) 2013-124452

(51) Int. Cl. G01B 9/02; A61B 3/10

(52) U.S. Cl. CPC G01B 9/02091; A61B 3/102
USPC 356/479; 351/206

(57) ABSTRACT

Provided is an optical tomography apparatus acquiring high-quality tomographic images even if light source characteristic is different between sweeps in first and second directions, when acquiring the image by reciprocating sweep. The optical tomography apparatus includes: a wavelength swept light source device; a splitting-combining unit splitting light from the device into measurement and reference light, and combining return light from an object to be measured by the measurement light and the reference light; and an image processing unit acquiring a tomographic image of the object based on combined light. The device includes a reciprocating sweep light source device performing a first wavelength sweep from short to long wavelength and a second wavelength sweep from long to short wavelength. The processing unit acquires first and second tomographic images based on signals acquired by these wavelength sweeps for the same site, and combines the tomographic images to acquire the tomographic image.

### BACKGROUND OF THE INVENTION

1. Field of the Invention

The present invention relates to an optical tomography apparatus, an optical tomography method, and an optical coherence tomography apparatus.

2. Description of the Related Art

A wavelength tunable (swept) light source is used in an inspection apparatus such as a laser spectroscopy apparatus, a dispersion measurement apparatus, a film thickness measurement apparatus, and a swept source optical coherence tomography (SS-OCT) apparatus. The SS-OCT is a technology to image a tomographic image of a subject to be inspected by using the optical coherence. This imaging technology can obtain a spatial resolution on the order of micrometers with non-invasiveness, and hence the technology has become an active area of research in the medical field in recent years. The SS-OCT is disclosed in Japanese Patent Application Laid-Open No. 2008-47730.

When configuring a medical imaging apparatus employing the SS-OCT technology, the time for acquiring an image can be shortened as a wavelength sweep rate is increased, and hence the wavelength sweep rate is one of the key parameters. On the other hand, it is desired that the SS-OCT apparatus have a capability to detect a structure deep inside the subject to be inspected, i.e., achieving a long coherence length. For this reason, a narrower oscillation spectral line width is desired as a factor for performance of a light source of the SS-OCT apparatus. Specifically, the coherence length L is defined by

L=λₒ²/nδλ (1)

where δλ is an oscillation spectral line width, λₒ is an oscillation wavelength, and n is a refractive index of the subject to be inspected. Therefore, the oscillation spectral line width needs to be decreased in order to broaden a measurement range in the depth direction of the subject to be inspected, which requires a wavelength swept light source having a narrow line width. In the meanwhile, as a light source that can achieve both of the fast wavelength sweep rate and the long coherence length, a wavelength sweep surface-emitting laser, which is obtained by combining a surface-emitting laser light source and a MEMS mirror, is getting attention. The wavelength swept surface-emitting laser is disclosed in Japanese Patent Application Laid-Open No. 2004-281733.

However, the wavelength swept surface-emitting laser has the following problems. That is, in such a wavelength swept surface-emitting laser, a stable light output is not obtained immediately after starting a drive from a drive-stopped state, and hence a delay is generated on a rising edge of the light output. In the surface-emitting laser, the internal temperature rise is relatively large at the time of drive, and the device characteristic thereof is sensitive to the temperature. Thus, the light output varies depending on the temperature even with the same current injection. Therefore, the light output at the rising time cannot be controlled with only the drive current.

A case where such a wavelength swept surface-emitting laser is used for an ophthalmic SS-OCT apparatus is described below. When a wavelength tunable surface-emitting laser obtained by combining a surface-emitting laser light source and a MEMS mirror is used in an SS-OCT apparatus, scanning and imaging a fundus by a reciprocating sweep may lead to image quality degradation. This is because the output is different between a case where the wavelength is swept from short wavelength to long wavelength and a case where the wavelength is swept from long wavelength to short wavelength, due to a nonlinear optical effect inside an active layer. The influence of this output difference causes a tomographic image to be different in contrast for every imaging point when the fundus is scanned and imaged by a reciprocating sweep, and as a result, the image quality is degraded.

On the other hand, a method of acquiring the tomographic image only by a unidirectional sweep is conceivable in order to obtain a high quality tomographic image in the SS-OCT apparatus using a wavelength swept light source. However, in the method of acquiring the tomographic image only by the unidirectional sweep, the light source ends up with being put the light out during a half of the time in the ophthalmic SS-OCT apparatus. This is because, in the ophthalmic SS-OCT apparatus, it is necessary to avoid emission of an unnecessary laser beam to the interior of the eye in order to prevent damage on the eye due to the laser beam.

In the wavelength tunable surface-emitting laser obtained by combining the surface-emitting laser light source and the MEMS mirror, this lights-off time is, for example, when the driving frequency of the MEMS mirror is 100 kHz, about 5 μs. The time constant of the temperature change of the surface-emitting laser is sub-μs to a few μs, and hence this lights-off time is enough to cause the output change due to the internal temperature change. Therefore, when the method of acquiring the tomographic image only by the unidirectional sweep is executed with the wavelength swept surface-emitting laser, there arises another problem in that the rising of the wavelength swept light output is delayed. In the case of a related-art wavelength swept light source device using an edge-emitting type gain medium, which has been used in the SS-OCT apparatus, the time constant of the temperature change due to light emission of the edge-emitting type gain medium is sufficiently large compared to the lights-off time, and hence there is no the above problem; however, the problem of the image quality degradation due to the reciprocating sweep is still remained.

### SUMMARY OF THE INVENTION

The present invention has been achieved in view of the above-mentioned problems, and it is an object of the present invention to provide an optical tomography apparatus and an optical tomography method, which can acquire a high quality tomographic image even if the light source characteristic is different between a sweep in a first direction and a sweep in a second direction that is opposite to the first direction, when acquiring the tomographic image by a reciprocating sweep by using a wavelength swept light source device.

According to one embodiment of the present invention, there is provided an optical tomography apparatus, including: a wavelength swept light source device; a splitting and combining unit configured to: split light emitted from the wavelength swept light source device into measurement light and reference light; and combine return light from an object to be measured by the measurement light and the reference light corresponding to the measurement light; and an image processing unit configured to perform image processing based on combined light obtained by combining the return light and the reference light to acquire a tomographic image of the object to be measured, in which: the wavelength swept light source device includes a reciprocating sweep type light source device configured to perform a first wavelength sweep from a short wavelength to a long wavelength and a second wavelength sweep from a long wavelength to a short wavelength in an alternate manner; and the image processing unit is configured to: acquire a first tomographic image generated based on a signal acquired by the first wavelength sweep and a second tomographic image generated based on a signal acquired by the second wavelength sweep for the same site of the object to be measured; and combine the first tomographic image and the second tomographic image to acquire the tomographic image of the object to be measured.

According to one embodiment of the present invention, there is provided an optical tomography method, including splitting light emitted from a wavelength swept light source device into measurement light and reference light, acquiring a tomographic image of an object to be measured based on light obtained by combining return light from the object to be measured by the measurement light and the reference light corresponding to the measurement light, the method comprising: performing a first wavelength sweep of light wavelength in one direction from a short wavelength to a long wavelength by the wavelength swept light source device; performing a second wavelength sweep of light wavelength in one direction from a long wavelength to a short wavelength by the wavelength swept light source device, the first wavelength sweep and the second wavelength sweep being performed in an alternate manner; generating a first tomographic image based on a signal acquired in the first wavelength sweep; generating a second tomographic image based on a signal acquired in the second wavelength sweep; and combining the first tomographic image and the second tomographic image to acquire the tomographic image of the object to be measured, in which the first wavelength sweep and the second wavelength sweep are performed at a same site of the object to be measured.

According to one embodiment of the present invention, there is provided an optical coherence tomography apparatus, including: a wavelength swept light source unit; an optical coherence system configured to: split light emitted from the wavelength swept light source unit into reference light and irradiation light to be radiated to an object to be measured; and generate interference light between reflected light of the irradiation light radiated to the object to be measured and the reference light; and an acquiring unit configured to acquire depth direction information of the object to be measured corresponding to a measurement point on a surface of the object to be measured, based on the interference light, in which: the wavelength swept light source unit is configured to perform a first wavelength sweep from a short wavelength to a long wavelength and a second wavelength sweep from a long wavelength to a short wavelength in an alternate manner; and the acquiring unit is configured to acquire the depth direction information of the object to be measured corresponding to the measurement point based on the interference light corresponding to the first wavelength sweep and the interference light corresponding to the second wavelength sweep.

Further features of the present invention will become apparent from the following description of exemplary embodiments with reference to the attached drawings.

### BRIEF DESCRIPTION OF THE DRAWINGS

FIG. 1 is a flowchart illustrating an operation of an optical tomography apparatus according to an embodiment of the present invention.

FIG. 2 is a diagram illustrating a configuration of an optical tomography apparatus according to an embodiment of the present invention.

FIG. 3 is a diagram illustrating a configuration example of an optical tomography apparatus according to Example 1 of the present invention.

FIG. 4 is a flowchart illustrating a tomographic image imaging procedure according to Comparative Example.

FIG. 5 is a graph showing wavelength swept spectrum according to Example 1 of the present invention.

FIG. 6 is a graph showing wavelength swept spectrum according to Comparative Example.

FIG. 7 is a diagram illustrating a wavelength swept light source used in Example 2 of the present invention.

FIG. 8 is a diagram illustrating a configuration example of an optical tomography apparatus according to Example 2 of the present invention.

FIG. 9 is a diagram illustrating an example of a wavelength tunable surface-emitting laser.

### DESCRIPTION OF THE EMBODIMENTS

Preferred embodiments of the present invention will now be described in detail in accordance with the accompanying drawings.

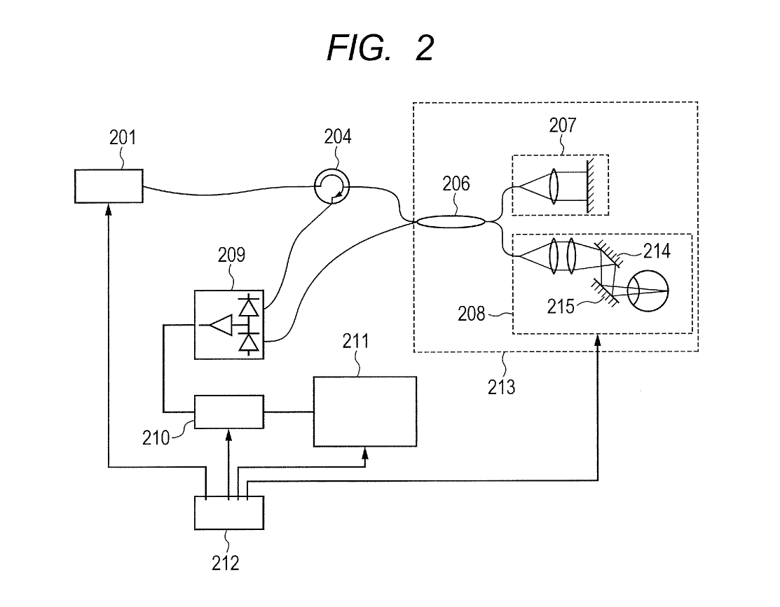

A configuration example of an OCT apparatus (optical tomography apparatus) and an optical tomography method according to an embodiment of the present invention is described below with reference to FIG. 2. The optical tomography apparatus (optical coherence tomography apparatus) according to this embodiment includes a wavelength swept light source device (light source unit), and is configured to split light emitted from the wavelength swept light source device into measurement light and reference light and to acquire a tomographic image of an object to be measured by performing image processing by an image processing unit (acquiring unit) based on light (interference light) obtained by combining reflected light from the object to be measured by the measurement light and the reference light corresponding to the measurement light.

The optical tomography apparatus includes a wavelength swept light source unit and an optical coherence system that splits light from the light source unit into reference light and irradiation light to be radiated to an object to be measured and generates interference light between reflected light of the irradiation light radiated to the object to be measured and the reference light. The optical tomography apparatus further includes an acquiring unit that acquires depth direction information of the object to be measured corresponding to a measurement point on a surface of the object to be measured, based on the interference light. The acquiring unit acquires the depth direction information of the object to be measured corresponding to the measurement point based on both of the interference light corresponding to the first wavelength sweep and the interference light corresponding to the second wavelength sweep. More specifically, the acquiring unit acquires the depth direction information of the object to be measured corresponding to same measurement point based on each of the interference light corresponding to the first wavelength sweep and the interference light corresponding to the second wavelength sweep and combines the acquired pieces of depth direction information of the object to be measured corresponding to the same measurement point. The information of the object to be measured in the depth direction can be acquired in a form of a tomographic image. In this case, the acquiring unit may acquire a first tomographic image from the interference light corresponding to the first wavelength sweep and a second tomographic image from the interference light corresponding to the second wavelength sweep and combine the first tomographic image and the second tomographic image. A combining process can be a simple addition process or a weight addition process of corresponding values or pixel values. Further, the combining process can be a simple averaging process or a weight averaging process of the corresponding values or pixel values. The optical tomography apparatus further includes a light receiving unit that receives the interference light and converts the received interference light into an electrical signal.

FIG. 2 illustrates an example of the optical tomography apparatus according to the present invention. As illustrated in FIG. 2, the optical tomography apparatus according to the present invention includes a wavelength swept light source 201. The wavelength swept light source 201 is, for example, a reciprocating sweep type wavelength swept light source that performs a first wavelength sweep from a short wavelength to a long wavelength and a second wavelength sweep from a long wavelength to a short wavelength in an alternate manner. For example, a MEMS-VCSEL type wavelength swept light source can be used as the wavelength swept light source 201, in which one resonator mirror of a vertical cavity surface-emitting laser is moved by a MEMS. Alternatively, a wavelength swept light source that performs a wavelength sweep by using a diffraction grating and Galvano Mirror, a light source that performs a wavelength sweep by using a diffraction grating and a MEMS mirror, a light source that performs a wavelength sweep by using a MEMS Fabry-Perot filter, a laser including a gain medium and an external resonator, or the like can be used as the wavelength swept light source 201. Further, the light source device emits the light from a time when the first wavelength sweep is started to a time when the next first wavelength sweep is started or from a time when the second wavelength sweep is started to a time when the next second wavelength sweep is started.

The output of the wavelength swept light source 201 passes through an optical circulator 204 and is split for a reference optical system 207 and a measurement optical system 208 by an optical coupler 206 (splitting and combining unit). Reflected light from the reference optical system 207 and reflected light or back-scattered light from the measurement optical system 208 enter the optical coupler 206 again, are interfered with each other, and are combined with each other. A portion 213 is an interferometer for acquiring an OCT signal. One part of interference light interfered in the optical coupler 206 is input to a differential detector 209 through the optical circulator 204, and the other part of the interference light is directly input to a differential detector 209 to be differentially detected. The light differentially detected by the differential detector 209 is converted into an electrical signal by the differential detector 209, and the electrical signal is converted into a digital signal by an analog-to-digital (AD) convertor 210. The digital signal is then subjected to a Fourier transform and various correction processing by a signal processing device (signal processing unit) 211 to acquire a tomographic image. The wavelength swept light source 201, the AD convertor 210, the signal processing device 211, and light beam scanning mechanisms (scanning mirrors) 214 and 215 in the measurement optical system are operated in synchronization with a signal from a control device 212.

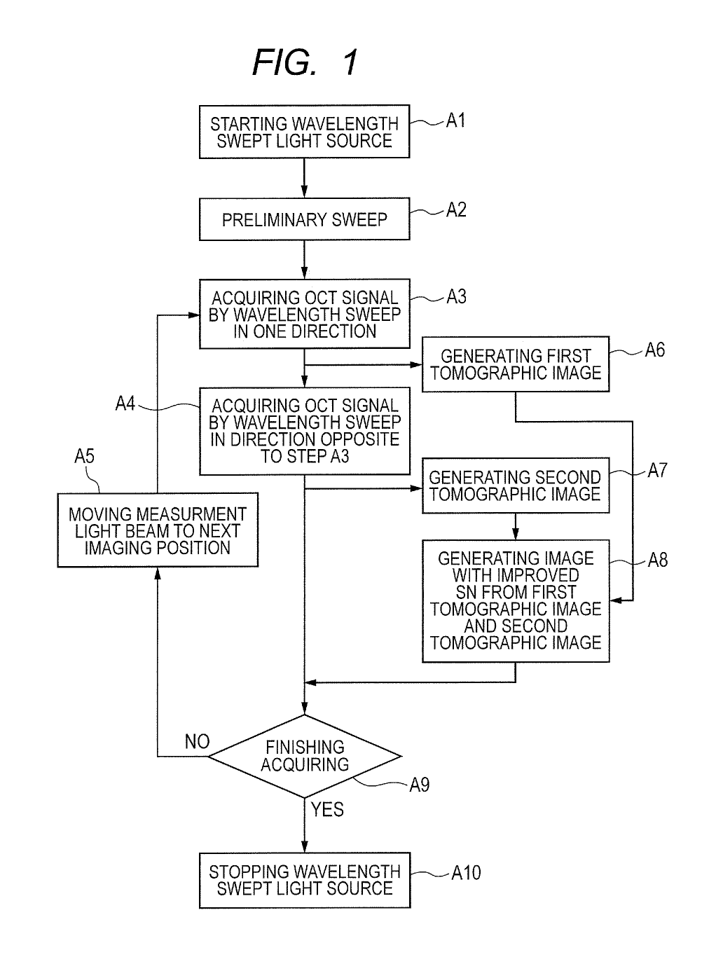

An operation of the OCT apparatus according to this embodiment when the object to be measured is an eye to be inspected is described in detail with reference to FIG. 1. In order to start acquiring the tomographic image, the wavelength swept light source is started first (Step A1). When the wavelength swept light source is a MEMS-VCSEL type light source, driving of a MEMS mirror in the wavelength swept light source 201 is started first, and then a current injection to a VCSEL (surface-emitting laser) is started to start the wavelength swept light source 201. In order to stabilize the output of the wavelength swept light source 201, at least one time of preliminary sweep is performed before acquiring the OCT signal (Step A2).

Next, the electrical signal corresponding to the interference light, which is detected by the differential detector 209 while the wavelength swept light source 201 performs the wavelength sweep in one direction from a short wavelength to a long wavelength or from a long wavelength to a short wavelength, is acquired by the AD convertor 210 and converted into the OCT signal (Step A3). The acquired OCT signal is subjected to a correction processing of linearizing wavenumber and a conversion process into a tomographic image by the signal processing device 211, to thereby generate a first tomographic image (Step A6). The generation of the first tomographic image can be performed while the wavelength swept light source 201 performs the wavelength sweep in a direction opposite to the wavelength sweep direction in Step A3. In other words, the step of generating the first tomographic image from the OCT signal acquired when the wavelength swept light source 201 performs the wavelength sweep from a short wavelength to a long wavelength may be performed during the step of acquiring the OCT signal by performing the wavelength sweep from a long wavelength to a short wavelength by the wavelength swept light source 201. Further, in contrast, the step of generating the first tomographic image from the OCT signal acquired when the wavelength swept light source 201 performs the wavelength sweep from a long wavelength to a short wavelength may be performed during the step of acquiring the OCT signal by performing the wavelength sweep from a short wavelength to a long wavelength by the wavelength swept light source 201.

Subsequently, in the similar manner as the first tomographic image, a second tomographic image is generated for the same site as the one for which the first tomographic image has been generated, while the wavelength swept light source 201 performs the wavelength sweep in a direction opposite to the wavelength sweep direction in Step A3 (Steps A4 and A7). While the OCT signal is acquired two times in Steps A3 and A4, the scanning mirrors 214 and 215 for scanning a fundus are stopped and the light beam for acquiring the tomographic image is stopped, and hence the first tomographic image and the second tomographic image in the same site can be obtained.

The two images, that is the first tomographic image and the second tomographic image, are combined, and the obtained tomographic image with the improved image quality is generated as a tomographic image of the imaging position (Step A8). An example of the combining process is a process of averaging the first tomographic image and the second tomographic image. The averaging process can be a simple averaging process or a weight averaging process. In the case of the latter, it is preferred to perform the weight averaging process by setting a weight of the first tomographic image having a higher SN ratio, which is generated based on the signal detected while performing the wavelength sweep from a short wavelength to a long wavelength, to be larger than a weight of the second tomographic image. This combining process can be any method so long as image processing of improving the image quality is performed, such as improving the SN ratio of the image or improving the dynamic range of the image. In this case, although a method of improving the SN ratio of the signal by processing the two signals in a state of being the acquired OCT signal can be considered, this method is difficult because a phase shift is likely to be generated between the signals in this method. In contrast to this, the method of processing the image after generating the two tomographic images as described in this embodiment is easy to process. After acquiring the tomographic image of one imaging point in the above manner, the scanning mirrors 214 and 215 in the measurement optical system 208 are driven to move the measurement light beam to the next imaging position (Step A5), and an acquisition of the tomographic image is performed in the similar manner. A three-dimensional tomographic image of the fundus can be acquired by repeating the above steps. After acquiring all imaging points (Step A9), the wavelength swept light source 201 is stopped (Step A10).

### EXAMPLES

Examples of the present invention are described below.

### Example 1

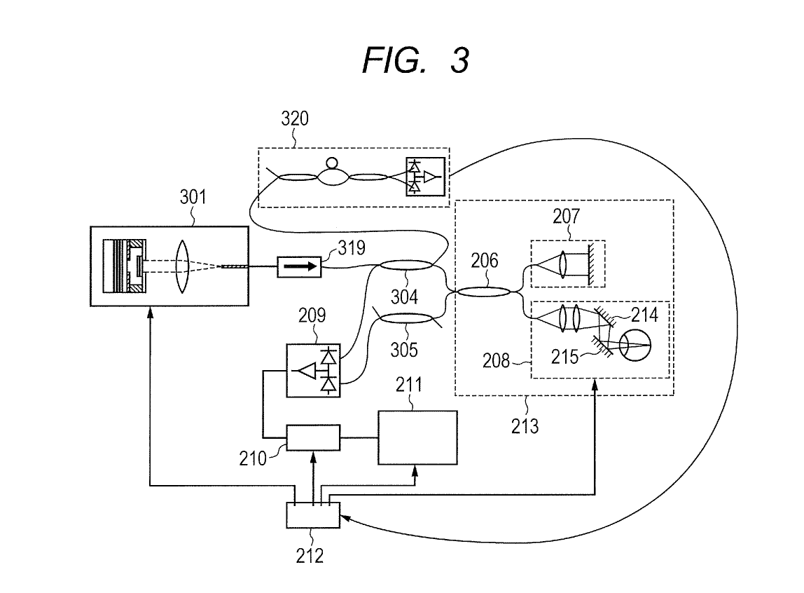

As Example 1, a configuration example of an optical tomography apparatus (OCT apparatus) and an optical tomography method to which the present invention is applied is described with reference to FIG. 3. The optical tomography apparatus illustrated in FIG. 3 includes a MEMS-VCSEL type wavelength swept light source 301 in which one resonant mirror of a vertical cavity surface-emitting laser is moved by the MEMS. The used light source emits light having a center wavelength of 850 nm and a wavelength sweep band of 60 nm.

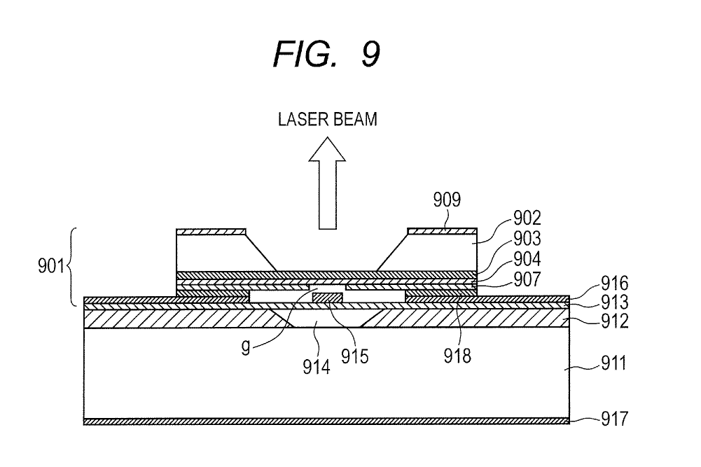

As such a wavelength tunable surface-emitting laser, a configuration illustrated in FIG. 9 is used. As illustrated in FIG. 9, a surface-emitting laser element 901 includes a GaAs substrate 902, a distributed Bragg reflector (DBR) layer 903, an active layer 904, and an upper electrode 909 and a lower electrode 907 for injecting electrical charges. The configuration illustrated in FIG. 9 further includes a Si substrate 911, a gap forming layer 912 for driving a mirror, a conductive layer 913 that also serves as a movable beam, a movable mirror 915, electrodes 916 and 917 for driving the movable mirror 915, and a bonding layer 918 for bonding the MEMS movable mirror 915 and a driven member of the surface-emitting laser element 901.

An operation of such a surface-emitting laser is described below. When a voltage is applied between the upper electrode 909 and the lower electrode 907, holes are injected from the lower electrode 907 to the active layer 904. At the same time, electrons are injected from the upper electrode 909 to the active layer 904 through the GaAs substrate 902 and the DBR layer 903. Light is emitted by combining the holes and the electrons in the active layer 904 that has the narrowest bandgap, and light of a desired wavelength is amplified by an optical resonator formed between the DBR layer 903 and the movable mirror 915. Then, the amplified light is emitted from the DBR layer 903 side. In this case, the wavelength of the emitted laser beam dependents on the size of an air gap g formed between the movable mirror 915 and the active layer 904, and hence the wavelength of the laser beam can be changed by changing the size of the air gap g. An air gap 914 is further formed between the Si substrate 911 and the conductive layer 913.

An operation of changing the wavelength of the laser beam is described below. When a driving voltage is applied between the electrodes 916 and 917, an electrostatic force is exerted between the conductive layer 913 and the Si substrate 911, and hence the movable mirror 915 on the movable beam 913 is displaced toward the Si substrate 911 side, such that the size of the air gap g is increased. Therefore, by controlling the size of the air gap g with control the driving voltage, a desired wavelength of the laser beam can be obtained.

In the OCT apparatus of FIG. 3, the output of a wavelength swept light source 301 passes through an optical isolator 319 and an optical coupler 304, and is split for the reference optical system 207 and the measurement optical system 208 at the optical coupler 206. In this example, the optical coupler 304 was used in lieu of the optical circulator. The reflected light from the reference optical system 207 and the back-scattered light from the measurement optical system 208 are interfered with each other at the optical coupler 206. A portion 213 is the interferometer for acquiring the OCT signal. The interference light interfered in the optical coupler 206 is distributed to the optical coupler 304 and an optical coupler 305, and output light beams from the optical couplers 304 and 305 are differentially detected by the differential detector 209.

The optical coupler 305 is disposed to take a balance of optical intensity of the interference light split from the optical coupler 304 and then input to the differential detector 209. An optical attenuator can be used in lieu of the optical coupler 305. Alternatively, the differential detector 209 can have a function of adjusting a balance of the differential input. The light differentially detected by the differential detector 209 is converted into an electrical signal, and the electrical signal is converted into a digital signal by the AD convertor 210. The digital signal is then subjected to the Fourier transform and various correction processing by the signal processing device 211 to acquire the tomographic image. Further, the output of the optical coupler 304 on a side that is not connected to the interferometer 213 is connected to a wavenumber clock generation device 320. The wavenumber clock generation device 320 includes a Mach-Zehnder interferometer and a differential detector. In this example, the optical pass length difference of the interferometer was set to 4 mm in order to obtain a wavenumber clock necessary to achieve an invasion depth length of 2 mm. The wavenumber clock generated by the wavenumber clock generation device 320 is input to the control device 212 as a data sampling clock. The wavelength swept light source 301, the AD convertor 210, the signal processing device 211, and the optical beam scanning mechanisms 214 and 215 in the measurement optical system are operated in synchronization with a signal from the control device 212.

By using the optical tomography apparatus (OCT apparatus) illustrated in FIG. 3 and disposing a silver mirror as an object to be measured in the measurement optical system 208, the image SN ratio of the apparatus was investigated. The tomographic image was acquired by the method of acquiring the tomographic image according to the present invention illustrated in FIG. 1, and the SN ratio of 96.1 dB was obtained. In contrast to this, the SN ratio of the first tomographic image generated in Step A6 in FIG. 1 was 94.7 dB and the SN ratio of the second tomographic image generated in Step A7 was 94.5 dB, which confirmed that the SN ratio of the tomographic image was improved by the present invention.

### Comparative Example

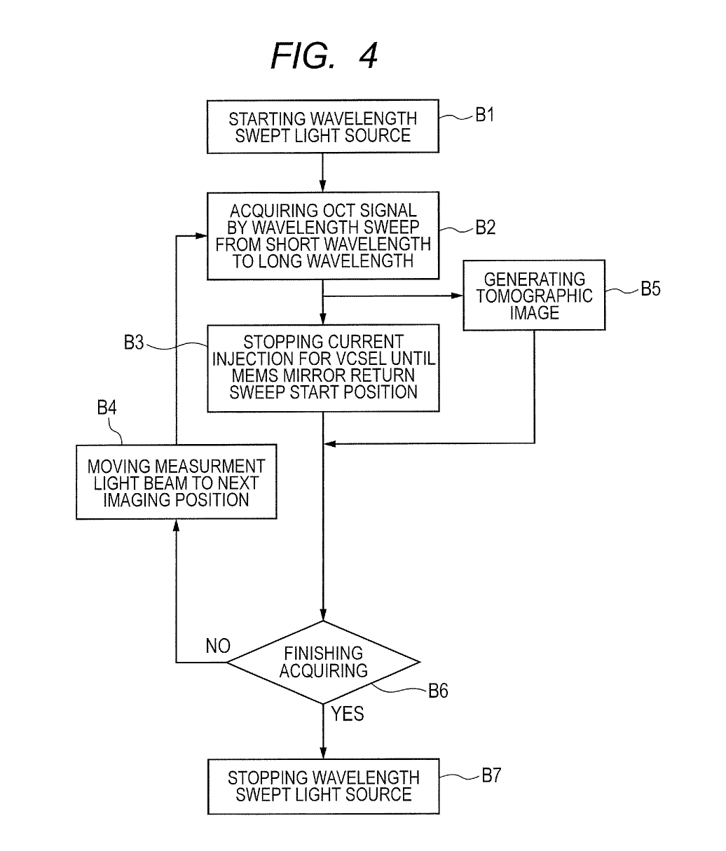

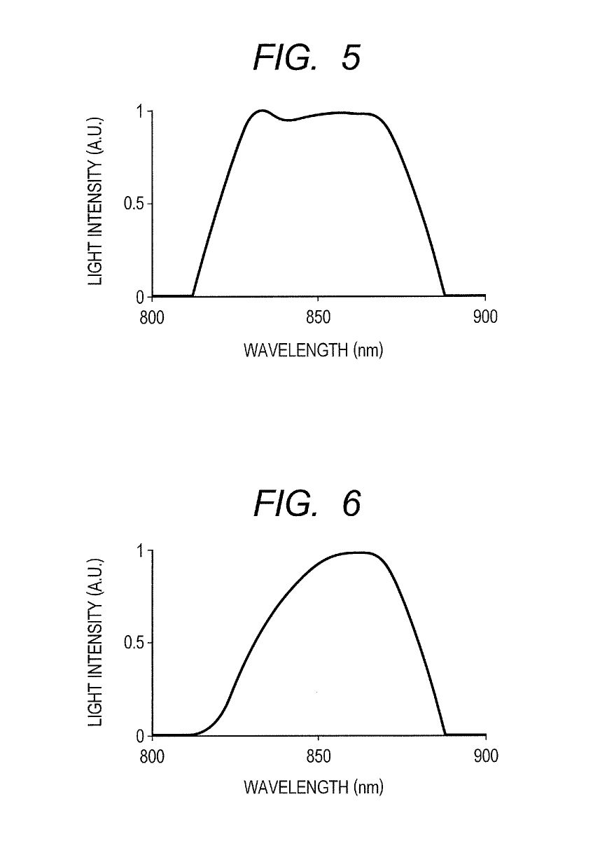

As Comparative Example, a tomographic image was acquired by an operation procedure illustrated in FIG. 4, which was similar to a tomographic image imaging procedure used in a related-art wavelength swept light source using an edge-emitting type light source, by using a wavelength swept light source 301 having a configuration illustrated in FIG. 3 which is similar to that used in Example 1. In order to confirm the effect of the present invention, wavelength swept spectrum of the wavelength swept light source under an operation was investigated by the procedures of Comparative Example and Example 1. As a result, the wavelength swept spectrum of the wavelength swept light source under an operation by the procedure of Comparative Example was shown in FIG. 6. In contrast to this, the wavelength swept spectrum when the wavelength swept light source under an operation by the procedure of Example 1 performed a wavelength sweep from a short wavelength to a long wavelength was shown in FIG. 5. The operation by the procedure of Comparative Example showed that the light amount on the short wavelength side, i.e., on a wavelength side where the wavelength sweep was started was decreased and the wavelength sweep band was decreased, which confirmed the effect of the present invention. The SN ratio of the image was investigated by imaging a silver mirror with the procedure of Comparative Example, in the similar manner to Example 1. The SN ratio thereof was 93.0 dB which was degraded by 1.8 dB compared to 94.8 dB of the SN ratio of the first tomographic image investigated in Example 1. In addition, a width of the tomographic image, which shows a surface of the silver mirror, in Comparative Example was broadened, which showed that the depth resolution was also degraded. This is because an effective band of the wavelength sweep was decreased due to the decrease of the light amount on the side where the wavelength sweep was started. As described above, it has been confirmed that, if the tomographic image imaging procedure used in the related-art wavelength swept light source using the edge-emitting type light source is adopted in the wavelength swept light source using the vertical cavity type light source, the SN ratio of the image and the depth resolution are degraded.

### Example 2

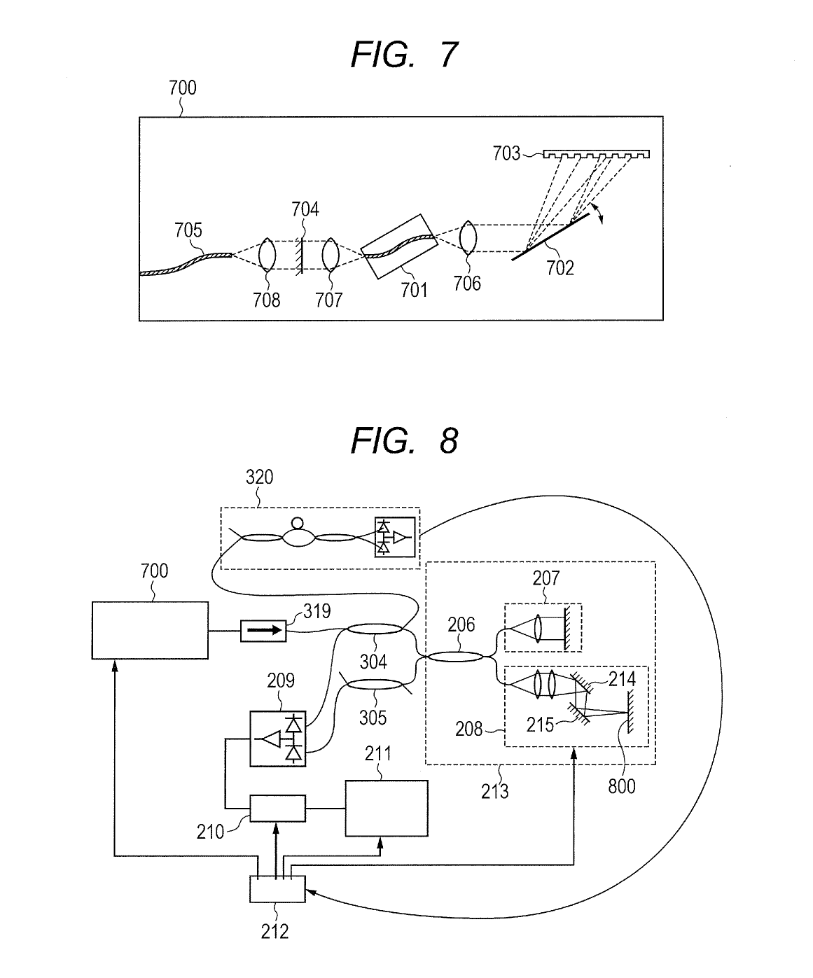

As Example 2, a configuration example of an optical tomography apparatus (OCT apparatus) that is different from Example 1 is described with reference to FIG. 8. As illustrated in FIG. 7, Example 2 is different from Example 1 in that a wavelength swept light source device using an edge-emitting type gain medium is used as a wavelength swept light source 700 in Example 2. The wavelength swept light source 700 used in Example 2 is described with reference to FIG. 7. As illustrated in FIG. 7, the wavelength swept light source 700 includes an edge-emitting type gain medium 701 having a center wavelength of 840 nm and an emission bandwidth of 40 nm. A MEMS mirror 702 has a mirror size of 1.8 mm by 1.8 mm and can deflect the light beam at 100 kHz with a deflection angle of 8 degrees. A reflection type diffraction grating 703 is a blazed diffraction grating with 2,200 lines/mm and a blaze wavelength of 860 nm. A half mirror 704 has a reflectivity of 10% and a transmissivity of 90%, and a pair of the half mirror 704 and the reflection type diffraction grating 703 constitute a resonator. Collimator lenses 706 and 707 produce a collimated beam having a diameter of 1.5 μm at 1/ê2. A coupling lens 708 and an output optical fiber 705 are disposed in the wavelength swept light source 700. The MEMS mirror 702 is deflected by ±2 degrees, so that the light beam enters the reflection type diffraction grating 703 at an angle of 63.75 degrees to 71.75 degrees, the wavelength swept light source 700 that performs the wavelength sweep with a center wavelength of 840 nm and a wavelength sweep bandwidth of 40 nm is constituted.

By using the OCT apparatus illustrated in FIG. 8 and disposing a silver mirror 800 as an object to be measured in the measurement optical system 208, the image SN ratio of the apparatus was investigated. The tomographic image was acquired by the method of acquiring the tomographic image according to the present invention illustrated in FIG. 1, and the satisfactory SN ratio of 95.8 dB was obtained. In contrast to this, the SN ratio of the first tomographic image generated in Step A6 in FIG. 1 was 94.7 dB and the SN ratio of the second tomographic image generated in Step A7 was 94.0 dB, which confirmed that the SN ratio of the tomographic image was improved by the present invention. As indicated in Example 2, it has been confirmed that the tomographic image acquiring method according to the present invention has an effect of improving the SN ratio of the tomographic image even when a wavelength swept light source using an edge-emitting type gain medium is applied to the SS-OCT.

According to one embodiment of the present invention, it is possible to achieve the optical tomography apparatus and the optical tomography method, which can acquire a high quality tomographic image even if the light source characteristic is different between a sweep in a first direction and a sweep in a second direction that is opposite to the first direction, when acquiring the tomographic image by a reciprocating sweep by using a wavelength swept light source device.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2013-124452, filed Jun. 13, 2013, which is hereby incorporated by reference herein in its entirety.

What is claimed is:

1. An optical tomography apparatus, comprising:
a wavelength swept light source device;
a splitting and combining unit configured to: split light emitted from the wavelength swept light source device into measurement light and reference light; and combine return light from an object to be measured by the measurement light and the reference light corresponding to the measurement light; and
an image processing unit configured to perform image processing based on combined light obtained by combining the return light and the reference light to acquire a tomographic image of the object to be measured, wherein:
the wavelength swept light source device comprises a reciprocating sweep type light source device configured to perform a first wavelength sweep from a short wavelength to a long wavelength and a second wavelength sweep from a long wavelength to a short wavelength in an alternate manner; and
the image processing unit is configured to: acquire a first tomographic image generated based on a signal acquired by the first wavelength sweep and a second tomographic image generated based on a signal acquired by the second wavelength sweep for the same site of the object to be measured; and combine the first tomographic image and the second tomographic image to acquire the tomographic image of the object to be measured.

2. The optical tomography apparatus according to claim 1, wherein the wavelength swept light source device comprises a surface-emitting laser.

3. The optical tomography apparatus according to claim 1, wherein the wavelength swept light source device comprises a light source device using an edge-emitting type gain medium.

4. The optical tomography apparatus according to claim 1, wherein the image processing unit is configured to perform a weight averaging process of the first tomographic image and the second tomographic image by setting a weight of the first tomographic image to be larger than a weight of the second tomographic image.

5. The optical tomography apparatus according to claim 1, wherein the wavelength swept light source device is configured to perform a preliminary sweep at least one time before acquiring the first tomographic image and the second tomographic image.

6. The optical tomography apparatus according to claim 1, wherein the object to be measured comprises an eye to be inspected.

7. An optical tomography method, comprising splitting light emitted from a wavelength swept light source device into measurement light and reference light, acquiring a tomographic image of an object to be measured based on light obtained by combining return light from the object to be measured by the measurement light and the reference light corresponding to the measurement light,
the method comprising:
performing a first wavelength sweep of light wavelength in one direction from a short wavelength to a long wavelength by the wavelength swept light source device;
performing a second wavelength sweep of light wavelength in one direction from a long wavelength to a short wavelength by the wavelength swept light source device, the first wavelength sweep and the second wavelength sweep being performed in an alternate manner;
generating a first tomographic image based on a signal acquired in the first wavelength sweep;
generating a second tomographic image based on a signal acquired in the second wavelength sweep; and
combining the first tomographic image and the second tomographic image to acquire the tomographic image of the object to be measured,
wherein the first wavelength sweep and the second wavelength sweep are performed at a same site of the object to be measured.

8. The optical tomography method according to claim 7, further comprising performing a preliminary sweep at least one time before the generating the first tomographic image and the second tomographic image.

9. The optical tomography method according to claim 8, wherein the wavelength swept light source device comprises a surface-emitting laser.

10. The optical tomography method according to claim 7, wherein the wavelength swept light source device comprises a light source device using an edge-emitting type gain medium.

11. The optical tomography method according to claim 7, wherein the combining comprises performing a weight averaging process of the first tomographic image and the second tomographic image by setting a weight of the first tomographic image to be larger than a weight of the second tomographic image.

12. The optical tomography method according to claim 7, wherein:
the generating the first tomographic image is performed during the second wavelength sweep; and
the generating the second tomographic image is performed during the first wavelength sweep.

13. The optical tomography method according to claim 7, wherein the object to be measured comprises an eye to be inspected.

14. An optical coherence tomography apparatus, comprising:
a wavelength swept light source unit;
an optical coherence system configured to: split light emitted from the wavelength swept light source unit into reference light and irradiation light to be radiated to an object to be measured; and generate interference light between reflected light of the irradiation light radiated to the object to be measured and the reference light; and
an acquiring unit configured to acquire depth direction information of the object to be measured corresponding to a measurement point on a surface of the object to be measured, based on the interference light, wherein:
the wavelength swept light source unit is configured to perform a first wavelength sweep from a short wavelength to a long wavelength and a second wavelength sweep from a long wavelength to a short wavelength in an alternate manner; and
the acquiring unit is configured to acquire the depth direction information of the object to be measured corresponding to the measurement point based on the interference light corresponding to the first wavelength sweep and the interference light corresponding to the second wavelength sweep.

15. The optical coherence tomography apparatus according to claim 14, wherein the wavelength swept light source unit is configured to emit the light one of from a time when the first wavelength sweep is started to a time when a next first wavelength sweep is started and from a time when the second wavelength sweep is started to a time when a next second wavelength sweep is started.

16. The optical coherence tomography apparatus according to claim 14, further comprising a light receiving unit configured to receive the interference light and convert the received interference light into an electrical signal.

17. The optical coherence tomography apparatus according to claim 14, wherein the acquiring unit is configured to acquire the depth direction information of the object to be measured by performing one of an addition process and an averaging process of first data indicating depth direction information of the object to be measured acquired based on the interference light corresponding to the first wavelength sweep and second data indicating depth direction information of the object to be measured acquired based on the interference light corresponding to the second wavelength sweep.

18. The optical coherence tomography apparatus according to claim 17, wherein the acquiring unit is configured to acquire the depth direction information of the object to be measured by performing one of a weight addition process and a weight averaging process of the first data and the second data by setting a weight of the first data to be larger than a weight of the second data.

19. The optical coherence tomography apparatus according to claim 14, wherein the wavelength swept light source unit comprises a surface-emitting laser.

20. The optical coherence tomography apparatus according to claim 14, wherein the wavelength swept light source unit comprises a light source unit using an edge-emitting type gain medium.
